SEQUENCE LISTING (2 sequences)

SEQ ID NO: 1 - protein, 123 aa, Artificial [sequence omitted]

SEQ ID NO: 2 - protein, 106 aa, Artificial [sequence omitted]

(19) United States
(12) Patent Application Publication
(10) Pub. No.: US 2024/0301512 A1
(43) Pub. Date: Sep. 12, 2024

(54) METHODS OF ASSESSING THE RISK OF DEVELOPING PROGRESSIVE MULTIFOCAL LEUKOENCEPHALOPATHY IN PATIENTS TREATED WITH VLA-4 ANTAGONISTS

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); CENTRE HOSPITALIER UNIVERSITAIRE DE TOULOUSE, Toulouse (FR); UNIVERSITE TOULOUSE III – PAUL SABATIER, Toulouse (FR)

(72) Inventors: Jacques AMAR, Toulouse (FR); David BRASSAT, Basel (CH); Béatrice PIGNOLET, Toulouse (FR)

(21) Appl. No.: 18/263,014

(22) PCT Filed: Jan. 28, 2022
(86) PCT No.: PCT/EP2022/052085

§ 371 Date: Jul. 26, 2023

(30) Foreign Application Priority Data
Jan. 29, 2021 (EP) 21305115.4

(51) Int. Cl. C12Q 1/689; C07K 16/28; C12Q 1/6883

(52) U.S. Cl. CPC C12Q 1/689; C07K 16/2842; C12Q 1/6883; C12Q 2600/106; C12Q 2600/172

(57) ABSTRACT

Natalizumab a monoclonal antibody is associated with the risk of progressive multifocal leukoencephalopathy (PML), an infection caused by the John Cunningham (JC) virus. The inventors explored the hypothesis that bacteria should be involved in the onset of PML in connection to the HLA-DR haplotype in multiple sclerosis (MS) patients. Thus 625 MS patients starting Natalizumab therapy from the BIONAT study were followed prospectively. Among those patients, 12 developed a PML. Outside the BIONAT cohort, we included nine additional MS patients with PML who had been referred to our center. For each patient, blood metagenomics sequencing and sequencing-based typing for HLA-DRB1*15:01 ancestral haplotype were determined. HLA-DRB1*15:01 haplotype carriers show a protection against PML (p=0.03). Among blood taxa, at genus level, Phyllobacterium was only significantly associated in HLA-DRB1*15:01 haplotype carriers with an inflammatory marker (p<0.0001) as opposed to HLA-DRB1*15:01 haplotype negative where no significant correlation was observed. Among the patients with no HLA-DRB1*15:01 haplotype, we showed a positive association (p=0.02) between the abundance of Phyllobacterium and PML whereas no significant association was observed in patients with HLA-DRB1*15:01 haplotype. JC positive virus patients with no HLA-DRB1*15:01 haplotype and a level of Phylobacterium in blood >2% have an odds ratio of 4.55 (95% confidence intervals 1.82-11.37; p=0.001) of developing or having PML under NTZ treatment. In conclusion, the inventors showed a relation between the HLA-DRB1*15:01 haplotype, the circulating microbiota and the risk of PML. The interaction between blood microbiota and the HLA-DRB1*15:01 haplotype may play a role in the virulence of the viruses.

### FIELD OF THE INVENTION

The present invention is in the field of medicine, and in particular in neurology and virology.

### BACKGROUND OF THE INVENTION

Multiple sclerosis (MS) is a major cause of disability in young adults (1,2). Natalizumab (NTZ), a monoclonal antibody that inhibits leukocyte migration across the blood-brain barrier has been approved for the treatment of Relapsing-Remitting Multiple Sclerosis, (3). However, NTZ is associated with the risk of progressive multifocal leukoencephalopathy (PML) development, an opportunistic brain infection that is caused by the John Cunningham (JC) virus (4). This opportunistic brain infection appears to occur after an interaction between the host and environmental factors (5) Regarding host factors, the role of HLA genes coding for MHC class II molecules merits analysis (6,7). First, the HLA class II allele HLA-DRB1*15:01 ancestral haplotype (8,9) are important MS susceptibility risk factors. Then, HLA class II genes play a pivotal role in the defense against pathogens (10). Regarding environmental factors potentially involved in the development of PML, several studies have now shown that the presence of specific bacterial species may promote viral infection. A large double-blind, placebo-controlled trial demonstrated that a pneumococcal conjugate vaccine not only reduced the incidence of pneumonia due to S. pneumoniae, but also prevented about 33% of pneumonia cases associated with respiratory viruses (11). Reciprocally, prior exposure to a commensal bacterium can elicit protection against a subsequent respiratory syncytial virus infection (12).

### SUMMARY OF THE INVENTION

The present invention is defined by the claims. In particular, the present relates to methods of assessing the risk of developing progressive multifocal leukoencephalopathy in patients treated with VLA-4 antagonists.

### DETAILED DESCRIPTION OF THE INVENTION

Natalizumab a monoclonal antibody is associated with the risk of progressive multifocal leukoencephalopathy (PML), an infection caused by the John Cunningham (JC) virus. The inventors explored the hypothesis that bacteria should be involved in the onset of PML in connection to the HLA-DR haplotype in multiple sclerosis (MS) patients. Thus 625 MS patients starting Natalizumab therapy from the BIONAT study were followed prospectively. Among those patients, 12 developed a PML. Outside the BIONAT cohort, they included nine additional MS patients with PML who had been referred to their center. For each patient, blood metagenomics sequencing and sequencing-based typing for HLA-DRB1*15:01 ancestral haplotype were determined. HLA-DRB1*15:01 haplotype carriers show a protection against PML (p=0.03). Among blood taxa, at genus level, Phyllobacterium was only significantly associated in HLA-DRB1*15:01 haplotype carriers with an inflammatory marker (p<0.0001) as opposed to HLA-DRB1*15:01 haplotype negative where no significant correlation was observed. Among the patients with no HLA-DRB1*15:01 haplotype, they showed a positive association (p=0.02) between the abundance of Phyllobacterium and PML whereas no significant association was observed in patients with HLA-DRB1*15:01 haplotype. JC positive virus patients with no HLA-DRB1*15:01 haplotype and a level of Phyllobacterium in blood >2% have an odds ratio of 4.55 (95% confidence intervals 1.82-11.37; p=0.001) of developing or having PML under NTZ treatment. In conclusion, the inventors showed a relation between the HLA-DRB1*15:01 haplotype, the circulating microbiota and the risk of PML. The interaction between blood microbiota and the HLA-DRB1*15:01 haplotype may play a role in the virulence of the viruses.

Accordingly, the first object of the present invention relates to a method of determining whether a patient has or is at risk of having a progressive multifocal leukoencephalopathy upon administration with a VLA-4 antagonist comprising determining the abundance of Genus Phyllobacterium in biological sample obtained from the patient wherein said abundance indicates whether the patient has or is at risk of having progressive multifocal leukoencephalopathy.

As used herein, the term “progressive multifocal leukoencephalopathy” or “PML” has its general meaning in the art and refers to a rare and often fatal viral disease characterized by progressive damage or inflammation of the white matter of the brain at multiple locations (multifocal). It is caused by the JC virus, which is normally present and kept under control by the immune system. The symptoms of PML are diverse, since they are related to the location and amount of damage in the brain, and may evolve over the course of several weeks to months. The most prominent symptoms are clumsiness; progressive weakness; and visual, speech, and sometimes personality changes. The progression of deficits leads to life-threatening disability and (frequently) death. Current diagnosis of PML can be made following brain biopsy or by combining observations of a progressive course of the disease, consistent white matter lesions visible on a magnetic resonance imaging (MRI) scan, and the detection of the JC virus in spinal fluid.

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(l−p) where p is the probability of event and (l−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

In some embodiments, the method herein described is applied to the patient after the treatment (i.e. the patient was administered with the VLA-4 antagonist). In some embodiments, the patient presents symptoms of PML without having undergone the routine screening to rule out all possible causes for PML. The methods described herein can be part of the routine set of tests performed on a subject who presents symptoms of PML such as clumsiness; progressive weakness; and visual, speech, and sometimes personality changes; The method of the present invention can be carried out in addition of other diagnostic tools that include magnetic resonance imaging.

In some embodiments, the method herein described is applied to the patient before the treatment (i.e. the patient was not yet administered with the VLA-4 antagonist).

In particular, the patient suffers from multiple sclerosis. As used herein the term “multiple sclerosis” has its general meaning in the art and refers to a demyelinating disorder of the central nervous system characterized, anatomically, by sclerotic plaques in the brain and spinal cord producing symptoms including (but not limited to) visual loss, diplopia, nystagmus, dysarthria, weakness, paresthesias, and bladder abnormalities. There are five distinct disease stages and/or types of MS, namely. (1) benign multiple sclerosis; (2) relapsing-remitting multiple sclerosis; (3) secondary progressive multiple sclerosis; (4) progressive relapsing multiple sclerosis; and (5) primary progressive multiple sclerosis.

In some embodiments, the present method comprises the step consisting of determining if the patient harbours a HLA-DR2 haplotype.

In particular, the patient harbours a HLA-DR2 haplotype. As used herein, the term “MHC Class II” refers to the human Major Histocompatibility Complex Class II proteins, binding peptides or genes. The human MHC region, also referred to as HLA, is found on chromosome six and includes the Class I region and the Class II region. Within the MHC Class II region are found the DP, DQ and DR subregions for Class II α chain and β chain genes (i.e., DPα, DPβ, DQα, DQβ, DRα, and DRβ). Thus the term “HLA-DR” refers to an MHC class II cell surface receptor encoded by the human leukocyte antigen complex on chromosome 6 region 6p21.31. In particular, HLA-DR2 (DR2) of the HLA-DR serotype system, is a broad antigen serotype that is now preferentially covered by HLA-DR15 and HLA-DR16 serotype group. The term “haplotype” is defined as a contiguous region of genomic DNA resulting from a non-random distribution of alleles on several gene loci of a same chromosome due to a low inter-chromosomal recombination in this particular region of the genome. As the MHC genes are proximal to each other on the chromosome, genetic recombination rarely occurs within the MHC and most individuals will inherit an intact set of parental alleles from each parent; such a set of linked genes is referred to as a haplotype, the MHC genes found in one haploid genome.

The HLA-DR2 haplotypes may be determined by any routine methods well known in the art that typically involved molecular typing of genomic DNA.

In some embodiments, the patient harbours a HLA-DR2 haplotype selected from the group consisting of DRB1*1501, DRB1*15021, DQA102 and the DW2 haplotypes.

As used herein, the term “VLA-4” has its general meaning in the art and refers to Integrin alpha4beta1 (Very Late Antigen-4), also known as CD49d/CD29. This integrin is an alpha/beta heterodimeric glycoprotein in which the alpha-4 subunit, named CD49d, is noncovalently associated with the beta-1 subunit, named CD29. The cell membrane molecule VCAM-1 (vascular cell adhesion molecule 1) and fibronectin (which is an extracellular matrix protein) bind to the integrin VLA-4, which can be normally expressed on leukocyte plasma membranes. The term may include naturally occurring VLA-4s and variants and modified forms thereof. The VLA-4 can be from any source, but typically is a mammalian (e.g., human and non-human primates) VLA-4, particularly a human VLA-4.

As used herein, the term “VLA-4 antagonist” has its general meaning in the art and includes any chemical or biological entity that, upon administration to a subject, results in inhibition or down-regulation of a biological activity associated with activation of the VLA-4 in the patient, including any of the downstream biological effects otherwise resulting from the binding to VLA-4 to its natural ligands (e.g. VCAM-1 or fibronectin). In general, VLA-4 antagonists are well known in the art, and comprise any agent that can block VLA-4 activation or any of the downstream biological effects of VLA-4 activation. For example, such a VLA-4 antagonist can act by occupying the binding site or a portion thereof of the VLA-4, thereby making the receptor inaccessible to its natural ligand (e.g. VCAM-1 or fibronectin) so that its normal biological activity is prevented or reduced. In the context of the present invention, VLA-4 antagonists are preferably selective for the VLA-4 as compared with the other VLA (VLA-1, VLA-2, VLA-3 and VLA-5). By “selective” it is meant that the affinity of the antagonist for the VLA-4 is at least 10-fold, preferably 25-fold, more preferably 100-fold, still preferably 500-fold higher than the affinity for other VLAs. The antagonistic activity of compounds towards the VLA-4 may be determined using various methods well known in the art. For example, the agents may be tested for their capacity to block the interaction of VLA-4 receptor cells bearing a natural ligand of VLA-4 (e.g. VCAM-1 or fibronectin), or purified natural ligand of VLA-4 (e.g. VCAM or fibronectin). Typically, the assay can be performed with VLA-4 and VCAM-1 expressed on the surface of cells, or with the VLA-4 mediated interaction with extracellular fibronectin or purified or recombinant VCAM-1.

In some embodiments, the VLA-4 antagonist may be a low molecular weight antagonist, e.g. a small organic molecule. Exemplary small organic molecules that are VLA-4 antagonists include but are not limited to those described in U.S. Pat. Nos. 6,407,06; 5,998,447; 6,034,238; 6,306,887; 6,355,662; 6,432,923; 6,514,952; 6,514,952; 6,667,331; 6,668,527; 6,794,506; 6,838,439; 6,838,439; 6,903,128; 6,953,802; 7,205,310; 7,223,762; 7,320,960; 7,514,409; 7,538,215 and in US Patent Application Publications Numbers US 2002/0049236; US 2002/0052470; US 2003/0087956; US 2003/0144328; US 2004/0110945; US 2004/0220148; US 2004/0266763; US 2005/0085459 US 2005/0222119; US 2007/0099921; US 2007/0129390; US 2008/0064720; US 2009/0048308; US 2009/0069376; US 2009/0192181 and US 2010/0016345 that are hereby incorporated by reference into the present disclosure.

In some embodiments, the VLA-4 antagonist according to the invention is a peptide. For example, the International Patent Application Publication No WO 96/01644 discloses peptides that inhibit binding of VLA-4 to VCAM-1. Other peptides, peptide derivatives or cyclic peptides that bind to VLA-4 and block its binding to VCAM-1 are described in WO 96/22966; WO 96/20216; U.S. Pat. No. 5,510,332; WO 96/00581 or WO 96/06108.

In some embodiments, the VLA-4 antagonist is an antibody (the term including antibody fragment) that can block VLA-4 activation. In particular, the VLA-4 antagonist may consist in an antibody directed against VLA-4 or a ligand of VLA-4 (e.g. VCAM-1 or fibronectin), in such a way that said antibody impairs the binding of said ligand to VLA-4. Monoclonal antibodies to the alpha-4 subunit of VLA-4 that block binding to VCAM-1 include HP2/1 (AMAC, Inc. Westbrook Me.), L25 (Clayberger et al., 1987), TY 21.6 (WO 95/19790), TY.12 (WO9105038) and HP2/4. Further antibodies binding to VLA-4 and blocking VCAM-1 binding are described in WO 94/17828. Humanized antibodies to alpha-4 integrin are described by in WO9519790. Another example of humanized monoclonal antibody directed to the alpha-4 subunit of VLA-4 is AN-100226 (Antegren) as described in Elices M J (1998) (Antegren Athena Neurosciences Inc. IDrugs. 1998 June; 1(2):221-7). Monoclonal antibodies that bind to VCAM-1 and block its interaction with VLA-4 are described in WO 95/30439. Other antibodies to VCAM-1 have been reported by Carlos et al., 1990 and Dore-Duffy et al., 1993. In some embodiments, said VLA-4 antibody is natalizumab that is a humanized antibody against VLA-4 as described in U.S. Pat. Nos. 5,840,299 and 6,033,665, which are herein incorporated by reference in their entireties. Natalizumab is a humanized IgG4[kappa] monoclonal antibody directed against the alpha4-integrins alpha4beta1 and alpha4beta7. In particular, the VH domain of natalizumab is as shown in SEQ ID NO:1 and the VL domain is as shown in SEQ ID NO:2.

SEQ ID NO: 1
QVQLVQSGAEVKKPGASVKVSCKASGFNIKDTYI
HWVRQAPGORLEWMGRIDPANGYTKYDPKFQGRV
TITADTSASTAYMELSSLRSEDTAVYYCAREGYY
GNYGVYAMDYWGQGTLVTVSS
SEQ ID NO: 2
DIQMTQSPSSLSASVGDRVTITCKTSQDINKYMA
WYQQTPGKAPRLLIHYTSALQPGIPSRFSGSGSG
RDYTFTISSLOPEDIATYYCLQYDNLWTFGQGTK
VEIK

As used herein, the term “biological sample” to any biological sample obtained from the purpose of evaluation in vitro. In some embodiments, the biological sample is a body fluid sample. Examples of body fluids are blood, serum, plasma, amniotic fluid, brain/spinal cord fluid, liquor, cerebrospinal fluid, sputum, throat and pharynx secretions and other mucous membrane secretions, synovial fluids, ascites, tear fluid, lymph fluid and urine. More particularly, the sample is a blood sample. As used herein, the term “blood sample” means a whole blood sample obtained from the patient.

As used herein, the term “Genus Phyllobacterium” has its general meaning in the art and refers to a genus of Gram-negative, oxidase- and catalase-positive, aerobic bacteria which is recognized as a separate taxon on the basis of ribosomal DNA homology and 16S rRNA data. Exemplary species include P. bourgognense, P. brassicacearum, P. catacumbas, P. endophyticum, P. ifriqiyense, P. leguminum, P. loti, P. myrsinacearum, P. sophorae and P. trifolii.

As used herein, the term “abundance” refers to the quantity or the concentration of said bacteria in a location/sample. In some embodiments, the abundance is absolute abundance. As used herein, the term “absolute abundance” refers to the concentration of said bacteria in a location/sample expressed for instance in number of UFC per mL or genome equivalent per mL. In some embodiments, the abundance is relative abundance. As used herein, the term “relative abundance” refers to the percent composition of a bacterium genus relative to the total number of bacteria genus in a given location/sample.

In some embodiments, the abundance of Genus Phyllobacterium bacteria is measuring by any routine method well known in the art and typically by using molecular methods. In some embodiments, the abundance of Genus Phyllobacterium is measuring using 16S rRNA deep-sequencing. In some embodiments, the abundance of Genus Phyllobacterium is measuring using the abundance table generated by the next-generation sequencing of 16S rRNA genes of all bacteria within a given biological sample using qPCR technique. Nucleic acids may be extracted from a sample by routine techniques such as those described in Diagnostic Molecular Microbiology: Principles and Applications (Persing et al. (eds), 1993, American Society for Microbiology, Washington D.C.). U.S. Pat. Nos. 4,683,202, 4,683,195, 4,800,159, and 4,965,188 disclose conventional PCR techniques. PCR typically employs two oligonucleotide primers that bind to a selected target nucleic acid sequence. Primers useful in the present invention include oligonucleotides capable of acting as a point of initiation of nucleic acid synthesis within the target nucleic acid sequence. qPCR involves use of a thermostable polymerase. Typically, the polymerase is a Taq polymerase (i.e. Thermus aquaticus polymerase). The primers are combined with PCR reagents under reaction conditions that induce primer extension. The newly synthesized strands form a double-stranded molecule that can be used in the succeeding steps of the reaction. The steps of strand separation, annealing, and elongation can be repeated as often as needed to produce the desired quantity of amplification products corresponding to the target nucleic acid sequence molecule. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times. The 16S deep-sequencing technique is well-described in the state of the art for instance, Shendure and Ji. “Next-generation DNA sequencing”, Nature Biotechnology, 26(10): 1135-1145 (2008)). The 16S deep-sequencing technique also known as “next-generation DNA sequencing” (“NGS”), “high-throughput sequencing”, “massively parallel sequencing” and “deep sequencing” refers to a method of sequencing a plurality of nucleic acids in parallel. See e.g., Bentley et al, Nature 2008, 456:53-59. The leading commercially available platforms produced by Roche/454 (Margulies et al, 2005a), Illumina/Solexa (Bentley et al, 2008), Life/APG (SOLID) (McKernan et al, 2009) and Pacific Biosciences (Eid et al, 2009) may be used for deep sequencing. For example, in the 454 method, the DNA to be sequenced is either fractionated and supplied with adaptors or segments of DNA can be PCR-amplified using primers containing the adaptors. The adaptors are nucleotide 25-mers required for binding to the DNA Capture Beads and for annealing the emulsion PCR Amplification Primers and the Sequencing Primer. The DNA fragments are made single stranded and are attached to DNA capture beads in a manner that allows only one DNA fragment to be attached to one bead. Next, the DNA containing beads are emulsified in a water-in-oil mixture resulting in microreactors containing just one bead. Within the microreactor, the fragment is PCR-amplified, resulting in a copy number of several million per bead. After PCR, the emulsion is broken and the beads are loaded onto a pico titer plate. Each well of the pico-titer plate can contain only one bead. Sequencing enzymes are added to the wells and nucleotides are flowed across the wells in a fixed order. The incorporation of a nucleotide results in the release of a pyrophosphate, which catalyzes a reaction leading to a chemiluminescent signal. This signal is recorded by a CCD camera and a software is used to translate the signals into a DNA sequence. In the Illumina method (Bentley (2008)), single stranded, adaptor-supplied fragments are attached to an optically transparent surface and subjected to “bridge amplification”. This procedure results in several million clusters, each containing copies of a unique DNA fragment. DNA polymerase, primers and four labeled reversible terminator nucleotides are added and the surface is imaged by laser fluorescence to determine the location and nature of the labels. Protecting groups are then removed and the process is repeated for several cycles. The SOLiD process (Shendure (2005)) is similar to 454 sequencing, DNA fragments are amplified on the surface of beads. Sequencing involves cycles of ligation and detection of labeled probes. Several other techniques for high-throughput sequencing are currently being developed. Examples of such are The Helicos system (Harris (2008)), Complete Genomics (Drmanac (2010)) and Pacific Biosciences (Lundquist (2008)). As this is an extremely rapidly developing technical field, the applicability to the present invention of high throughput sequencing methods will be obvious to a person skilled in the art.

In some embodiments, the method herein described comprises the steps consisting of comparing the determined abundance with a predetermined reference value wherein differential between said determined abundance and said predetermined reference value indicates whether or not the patient has or is at risk of having a PML.

In some embodiments, the method herein discloses comprises the steps consisting of comparing the determined abundance with a predetermined reference value and concluding that the patient has or is at risk of having a PML when the level determined at step i) is higher than the predetermined reference value.

In some embodiments, the method herein disclosed comprises the steps consisting of comparing the determined abundance with a predetermined reference value and concluding that the patient has or is at risk of having a PML when the level determined at step i) is higher than the predetermined reference value and when the patient does not harbour a DRB1*1501 haplotype.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the abundance of Genus Phyllobacterium in a group of reference, one can use algorithmic analysis for the statistic treatment of the abundances determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the method herein disclosed relates to a method of determining whether a patient has or is at risk of having a progressive multifocal leukoencephalopathy upon administration with Natalizumab comprising i) determining the abundance of Genus Phyllobacterium in biological sample obtained from the patient, determining if the patient harbours a DRB1*1501 haplotype, comparing the determined abundance with a predetermined reference value and concluding that the patient has or is at risk of having a PML when the level determined at step i) is higher than the predetermined reference value and when the patient does not harbour a DRB1*1501 haplotype.

In some embodiments, the method herein disclosed comprises a step consisting of calculating a score that is compared to a predetermined reference value wherein a difference between said score and said reference value indicates whether the patient has or is at risk of having a PML.

As used herein, the term “score” refers to a piece of information, usually a number that conveys the result of the patient on a test. A risk scoring system separates a patient population into different risk groups; herein the process of risk stratification classifies the patients into very high-risk, high-risk, intermediate-risk and low-risk groups. Typically, the score is based on the abundance of Genus Phyllobacterium and may typically include additional risk factors, such as presence or absence of some HLA-DR2 haplotypes, duration of treatment with the VLA-4 antagonist (e.g. natalizumab), presence or absence of anti-John Cunningham virus antibodies, and previous treatment with immunosuppressants. Based on the above input features obtained from the patient, an operator can calculate a numerical function of the above list of inputs by applying an algorithm. For instance this numerical function may return a number, i.e. score (R), for instance between zero and one, where zero is the lowest possible risk indication and one is the highest. This numerical output may also be compared to a threshold (T) value between zero and one. If the risk score exceeds the threshold T, it is meant than the patient has or is at risk of having a PML and if the risk score is under the threshold T, it is meant than the patient has not or is not at risk of having a PML.

In some embodiments, the method herein disclosed comprises the use of a classification algorithm.

As used herein, the term “algorithm” is any mathematical equation, algorithmic, analytical or programmed process, or statistical technique that takes one or more continuous parameters and calculates an output value, sometimes referred to as an “index” or “index value.” Non-limiting examples of algorithms include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining parameters are linear and non-linear equations and statistical classification analyses to determine the relationship between levels of said parameters and the risk of allograft loss. Of particular interest are structural and syntactic statistical classification algorithms, and methods of risk index construction, utilizing pattern recognition features, including established techniques such as cross-correlation, Principal Components Analysis (PCA), factor rotation, Logistic Regression (Log Reg), Linear Discriminant Analysis (LDA), Eigengene Linear Discriminant Analysis (ELDA), Topological Data Analysis (TDA), Neural Networks, Support Vector Machine (SVM) algorithm and Random Forests algorithm (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, Recommender System Algorithm and Hidden Markov Models, among others. Other techniques may be used in survival and time to event hazard analysis, including Cox, Weibull, Kaplan-Meier and Greenwood models well known to those of skill in the art.

In some embodiments, the method of the present invention comprises the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting (e.g. XGBoost). Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models. The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering. In some instances, the machine learning algorithms comprise a reinforcement learning algorithm Examples of reinforcement learning algorithms include, but are not limited to, temporal difference learning, Q-learning and Learning Automata. Alternatively, the machine learning algorithm may comprise Data Pre-processing. In some embodiments, the boosting model includes the XGBoost algorithm.

A further object of the present invention relates to a kit or device for carrying out the method herein disclosed. In some embodiments, the kits or devices of the present invention comprise at least one sample collection container for sample collection. Collection devices and container include but are not limited to syringes, lancets, BD VACUTAINER® blood collection tubes. In some embodiments, the kits or devices described herein further comprise instructions for using the kit or device and interpretation of results. In some embodiments, the kit or device of the present invention further comprises a microprocessor to implement an algorithm so as to determine the probability that the patient has a PML. In some embodiments, the kit or device of the present invention further comprises a visual display and/or audible signal that indicates the probability determined by the microprocessor.

Once the patient is identified as having or being at risk of having a PML, the physician can decide further therapeutic options. Prognosis of PML is poor, since no specific therapy is available. Thus in the absence of any therapy, it would be particularly helpful to be able to predict the risk whether the patient has or is at risk of having a PML. Hence, there exists a need for means to determine at an early stage, i.e. during initial diagnosis of a disease or condition potentially treatable with a VLA-4 antagonist known to run a risk of triggering PML, or even before the onset of such disease or condition, whether such the patient is likely to suffer from PML. The method herein disclosed is thus particularly advantageous to monitor t patients receiving or expected to receive a VLA-4 antagonist thus to avoid the possible development of PML or even another complication at a later stage.

A further object of the present invention relates to a method of therapy of a patient with a VLA-4 antagonist comprising i) determining whether the patient has or is at risk of having a PML and ii) administering to the patient a therapeutically effective amount of the VLA-4 antagonist when it is concluded that the patient has not or is not at risk of having a PML.

A further object of the present invention relates to a method of therapy of a patient with a VLA-4 antagonist comprising i) determining whether the patient has or is at risk of having a PML and ii) discontinuing the therapy with the VLA-4 antagonist when it is concluded that that the patient has not or is not at risk of having a PML.

The invention will be further illustrated by the following figures and examples. However, these examples and figures should not be interpreted in any way as limiting the scope of the present invention.

### FIGURES

FIG. 1: Relative abundance (%) of Phyllobacterium in blood and PML in JC virus-positive patients. FIG. 1A: JC Virus-positive patients with no haplotype DR2. FIG. 1B: JCV-positive patients with no haplotype DR2. PML: progressive multifocal leukoencephalopathy

FIG. 2 Distribution of the relative abundance of Genus Phyllobacterium and PML according to DR2 status. FIG. 2A JCV-positive patients with haplotype DR2. FIG. 2B: JCV-positive patients with no Haplotype DR2. PML: progressive multifocal leukoencephalopathy

### EXAMPLE

### Methods

### Population

The study population has been described in detail elsewhere (13) (BIONAT cohort, ClinicalTrials.gov identifier: NCT00942214). Briefly, patients with relapsing remitting MS starting NTZ therapy at 18 MS centers in France as of June 2007 were included and were followed prospectively. All patients gave their written informed consent. All patients were followed up clinically and underwent brain magnetic resonance imaging (MRI) just before initiation of the treatment and after completion of 1 and 2 years of treatment. Biological samples, including serum, were collected at baseline before the first NTZ infusion, at 1 year and 2 years of NTZ. Anti-JCV antibody (Ab) prevalence was measured in all analyzed patients with the first generation two-step validated ELISA test at baseline. Among those patients, 12 patients developed a PML. Outside the BIONAT cohort, we included nine additional patients that has been referred to our center. We refer to those patients as Beyond BestMS PML (BBMS/ClinicalTrials.gouv, Identifier NCT01981161) case series;

In this study, the diagnosis of PML was confirmed by one of two sets of criteria: Brain tissue (from biopsy or postmortem examination) showing evidence of viral cytopathic changes on hematoxylin and eosin staining associated with either positive immunohistochemistry for SV40 or in situ hybridization for JCV DNA or PCR detection of JCV DNA in CSF or in brain biopsy specimens, preferably by ultrasensitive quantitative PCR testing (limit of quantification of ≤50 copies/mL), along with a detailed description of brain MRI findings consistent with PML (14).

16SrDNA Quantitation by Real-Time qPCR and Profiling by Directed Metagenomics Sequencing

For each patient, serum samples were collected and store at −80° C. until DNA extraction. Total DNA was extracted from 200 μl of sera using the NucleoSpin® Blood kit (Macherey-Nagel, Germany) after a mechanical lysis step of 2×30 sec at 20 Hz in a bead beater (TissueLyser, Qiagen, Netherlands) with 0.1 mm glass beads (MoBio, USA). The quality and quantity of extracted DNA were controlled by gel electrophoresis (1% w/w agarose in TBE 0.5×) and absorbance spectroscopy (NanoDrop 2000 UV spectrophotometer; Thermo Scientific, USA).

The V3-V4 hyper-variable regions of the 16S rDNA were quantified by qPCR, sequenced with the MiSeq technology (Illumina, USA) and clustered into OTU before taxonomic assignment as described previously (15-17).

### Statistical Analysis

Continuous variables were recorded as medians and 25th to 75th percentiles and categorical variables as percentages. We used the Chi2 test for categorical variables and the Student's t-test or the Wilcoxon tests for continuous variables as appropriate according to the skewness of the distribution as assessed by the Shapiro-Wilk's test; p<0.05 was considered as statistically significant. A logistic regression analysis was carried out to identify the predictive value of blood taxa found to be associated in interaction with HLA-DRB1*15:01 haplotype both with inflammatory markers and the risk of developing or having PML. The identification of blood taxa at the genus level associated with inflammatory markers was performed using correlation analysis (Spearman test). Because of the large number of correlations, p<0.0001 was considered as statistically significant in this set of correlation analysis. Statistical analyses were performed using SAS software (version 9.4 for Windows).

### Results

The characteristics of the study population are described in Table 1. PML was diagnosed only in JC virus-positive patients. HLA-DRB1*15:01 haplotype carriers showed a significant protection against PML. Indeed, the HLA-DRB1*15:01 haplotype was observed more frequently in patients that developed PML (from the BIONAT cohort) or with previously diagnosed PML (BBMS case series) (p=0.03) (Table 2). Blood metagenome was analyzed in all patients who develop PML (from BIONAT, and Beyond Best-MS). Due to technical problems, 5 out of 625 patients (0.08%) from the BIONAT cohort have been excluded (none of them with PML).

### Response to Genus Phyllobacterium Varies According to the Presence of the Haplotype DR2

Among blood taxa, genus Phyllobacterium only was significantly associated in HLA-DRB1*15:01 ancestral haplotype carriers with an inflammatory marker (Table 3) as opposed to HLA-DRB1*15:01 ancestral haplotype negative where no significant correlation was observed between any bacterial Genus and inflammatory markers. Further in HLA-DRB1*15:01 haplotype carrier patients, the relative abundance of Genus Phyllobacterium at baseline before NTZ treatment, tended to be correlated with baseline CD8 T lymphocyte count (r=0.15; p=0.02) and was significantly and positively correlated with the number of CD8 T lymphocytes at one year follow up (r=0.40; p<0.0001) (Table 3).

No HLA-DRB1*15:01 Ancestral Haplotype and Genus Phyllobacterium are Associated with PML Development

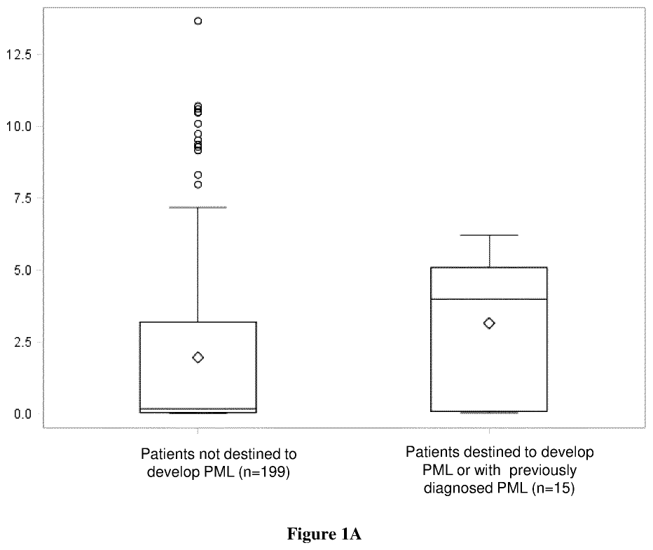

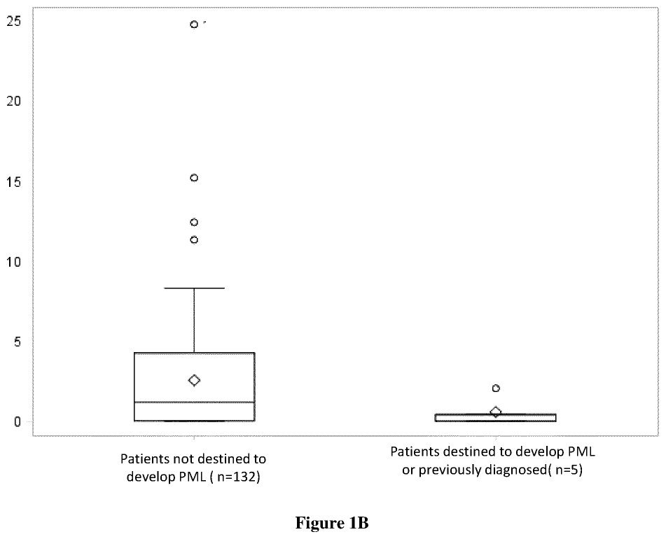

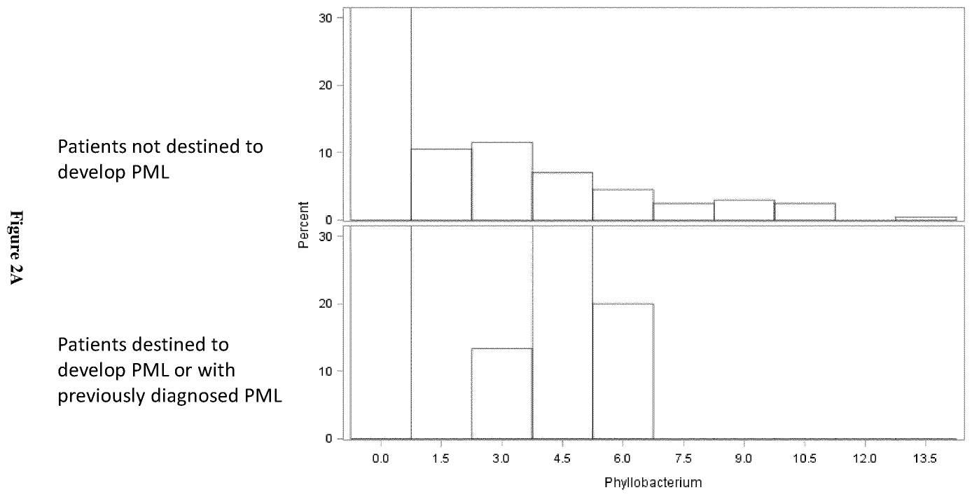

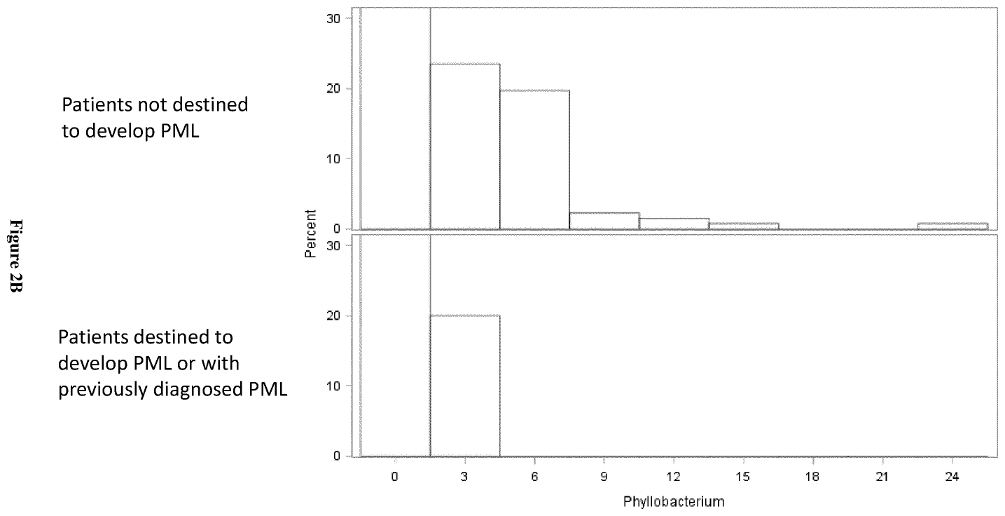

Among the JC virus-positive patients with no HLA-DRB1*15:01 haplotype, we showed a significant and positive association (p=0.02) between the abundance of Genus Phyllobacterium and the presence and/or the development of PML (FIG. 1A) whereas no significant association was observed in patients with HLA-DRB1*15:01 haplotype (FIG. 1B). In addition, in JC virus-positive patients with no HLA-DRB1*15:01 haplotype who developed PML, we observed a bimodal distribution of Genus Phyllobacterium (FIG. 2A) in contrast to patients not destined to develop PML and patients with HLA-DRB1*15:01 haplotype whether or not they develop PML (FIG. 2B). Eventually, JC-positive virus patients with no HLA-DRB1*15:01 haplotype and baseline level of Genus Phylobacterium in blood >2% had an odds ratio of 4.55 (95% confidence intervals (CI) 1.82-11.37; p=0.001) of developing or having PML under NTZ treatment.

### DISCUSSION

The main finding of this study is about HLA-DRB1*15:01 haplotype carriers and the abundance of 16S rDNA belonging to Genus Phyllobacterium in blood interact together to influence this risk of PML: the higher the abundance of Genus Phyllobacterium in blood the higher is the risk of developing PML in HLA-DRB1*15:01 haplotype non DR2 carrier patients (odds ratio for a relative abundance of the Genus Phyllobacterium in serum >2 percent: 4.55 95% CI (1.82-11.37)).

What is the mechanism behind this interaction? We did not observe a correlation between the abundance Genus Phyllobacterium in blood at baseline and CD8 T lymphocytes count at one-year follow-up after the initiation of NTZ in patients with no HLA-DRB1*15:01 haplotype whereas this correlation was present in carriers only. Importantly, molecules encoding by HLA-DRB1*15:01 haplotype are MHC class II molecules that are specialized for the presentation of antigens to T lymphocytes (10). In this respect, MHC class II molecules are expressed constitutively on antigen-presenting cells of the immune system only. In addition, human T lymphocytes express MHC class II molecules following activation. Therefore, the strong correlation only observed in patients with DR2 carrier between the abundance of the Genus Phyllobacterium and CD8 T lymphocytes at one year follow-up after the initiation of NTZ suggests that the immune system's response to the DNA belonging to Genus Phyllobacterium varies according to the presence of the haplotype DR2. It is noteworthy that allelic variations of the genes coding for MHC class II molecules have been associated with susceptibility to bacterial infection (18-20). Also, Genus Phyllobacterium which is a Genus of environmental bacteria isolated for example in plant roots (21) has been observed in various human diseases. To illustrate a higher level of Genus Phyllobacterium has been reported in gut microbiota in infants with hepatic disease (22), airway microbiota from patients with chronic obstructive pulmonary disease (23), patients with cystic fibrosis (24,25) and in the gastric microbial community from patients with gastric cancer (26). Furthermore, the presence of Phyllobacterium has been observed from pediatric kidney stones (27) or in the gut from preterm infants hospitalized in neonatal intensive care units (28). Moreover, this Genus has been involved in the development of bacillary angiomatosis in immunocompromised HIV-infected patients with a markedly low level of CD4 T lymphocytes (29). Considering these data, the current results suggest that the continuous work of the immune system to control these environmental bacteria is influenced by HLA-DRB1*15:01 ancestral haplotype and that the MHC class II-driven immune response pattern to the Genus Phyllobacterium should be a risk factor for the development of PML in NTZ-treated patients. Indeed, it has been shown that bacteria can facilitate enteric virus co-infection of mammalian cells (30,31) and it has been observed that JC Virus whose DNA is frequently present in the upper and lower gastrointestinal tract of healthy adults (32,33) can infect the myenteric plexuses in patients with intestinal disease (34). In this respect, an increasing body of work highlights how bacterial populations may contribute to infection (35). For example, both poliovirus and norovirus provide examples of viruses with enhanced pathogenesis when directly binding commensal enteric bacteria (36,37). These data suggest that since NTZ limits the ability of leukocytes to firmly adhere to endothelial surfaces then cross the endothelium and enter the mucosa, it may thereby influence the intestinal defense against Genus Phyllobacterium and then the development of PML in genetically predisposed patients. Is our finding of translational medicine important? Currently, the risk of PML can be quantified according to an algorithm that incorporates the following 3 risk factors: (1) duration of NTZ treatment, (2) presence of anti-John Cunningham virus antibodies, and (3) previous treatment with immunosuppressants. Risk mitigation procedures have been proposed based on the JC virus (38). Applying these procedures allows to decrease PML incidence (39). However, there is a room for improving these procedures for the MS Patients benefit (40). In this context, additional biomarkers are needed in clinical practice to identify patients at greater risk for PML. In this respect, we observed that patients with no haplotype DR2 and detectable Genus Phyllobacterium in blood had an increasing risk of PML. Thus, the current results suggest testing in a validation cohort the predictive role of both HLA-DRB1*15:01 haplotype and Genus Phyllobacterium.

In conclusion, our study shows for the first time a relation between the HLA-DRB1*15:01 ancestral haplotype, the circulating microbiota and the risk of John Cunningham virus infection of the brain. These relations are probably driven by an MHC class II-driven immune response pattern required to control commensals. These data suggest that the interaction between blood microbiota and the HLA-DRB1*15:01 ancestral haplotype may play a role in the virulence of the viruses.

### TABLES

TABLE 1
Characteristics of the Best MS population

| | Patients not | | Patients who | |
|---|---|---|---|---|
| | developing PML | | develop PML | |
| N | 613 | | 12 | |
| Female gender (%) | 455 | (74.23) | 10 | (83.33) |
| Age (y) | 37 | (30-44) | 39 | (36-48) |
| Age at MS onset (y)^{1, a} | 27 | (22-34) | 26 | (18-32) |
| Age at treatment start (y) | 36 | (29-43) | 39 | (36-48) |
| EDSS at T0 ^{2, a} | 3.0 | (2.0-4.5) | 4.0 | (3.0-5.5) |
| MSSS at T0 ^{3, a} | 4.60 | (2.70-6.81) | 4.55 | (3.83-6.33) |
| Lymphocyte count at T0 (10⁻³/mm³) ^{(4), a} | 1.80 | (1.44-2.34) | 1.70 | (1.30-2.10) |
| Previous Treatment ^{a} | | | | |
| Immunomodulatory agent n(%) | 418 | (68.19) | 9 | (81.82) |
| Immunomodulatory plus | 126 | (20.55) | 2 | (18.18) |
| immunosuppressive agents n(%) | | | | |
| Immunosuppressive agent n(%) | 12 | (1.96) | 0 | |
| naïve n(%) | 57 | (9.30) | 0 | |
| EDSS at T1 ^{5, a} | 2.5 | (1.5-4) | 4.0 | (2.0-6.0) |
| MSSS at T1 ^{6, a} | 3.79 | (1.77-5.64) | 4.94 | (1.28-6.56) |
| IgG at T0 ^{7, a} | 9.78 | (8.31-11.40) | 9.98 | (8.88-12.6) |
| IgA at T0 ^{7, a} | 1.98 | (1.48-2.50) | 1.93 | (1.40-2.28) |
| IgM at T0 ^{8, a} | 1.20 | (0.88-1.63) | 1.07 | (0.65-1.48) |
| IgG at T1 ^{9, b} | 9.26 | (7.58-10.90) | 9.38 | (8.76-9.74) |
| IgA at T1 ^{9, b} | 1.78 | (1.36-2.30) | 1.46 | (1.32-1.62) |
| IgM at T1 ^{9, b} | 0.69 | (0.49-0.97) | 0.43 | (0.34-0.64) |
| Neutrophils at T0 ^{10, c} | 4.0 | (3.0-5.3) | 4.3 | (3.6-4.5) |
| Lymphocytes at T0 ^{11, a} | 1.80 | (1.44-2.34) | 1.70 | (1.30-2.10) |
| T lymphocytes CD4 at T0 ^{12, a} | 863.5 | (683.0-1131.0) | 992 | (748-1097) |
| T lymphocytes CD8 at T0 ^{13, a} | 391 | (292-554) | 312 | (212-567) |
| T lymphocytes CD19 at T0 ^{14, d} | 237 | (175-336) | 242.5 | (198.0-291.0) |
| Lymphocytes at T1 ^{15, b} | 3.28 | (2.66-3.99) | 2.96 | (2.90-3.49) |
| T lymphocytes CD4 at T1 ^{16, b} | 1365 | (1138-1728) | 1287.5 | (1061.0-1700.0) |
| T lymphocytes CD8 at T1 ^{16, b} | 725 | (548-943) | 808.5 | (591.5-929.0) |
| T lymphocytes CD19 at T1 ^{17, b} | 732 | (564-964) | 680.0 | (581.5-756.0) |
| Positive JC virus antibodies status ¹⁸ | 393 | (65.83) | 12 | (100) |

Number in parentheses of missing data in patients not destined to develop a PML: ¹(2); ² (3); ³ (23); ⁽⁴⁾ (75); ⁵ (12), ⁶ (24); ⁷ (79); ⁸ (81); ⁹ (403); ¹⁰ (136); ¹¹ (75); ¹² (61); ¹³ (62); ¹⁴ (142); ¹⁵ (409); ¹⁶ (386), ¹⁷ (392) , ¹⁸ (17)
Number in parentheses of missing data in patients destined to develop a PML: ^{a} (1); ^{b} (8); ^{c} (3); ^{d} (2);

TABLE 2
HLA-DRB1*15:01 ancestral haplotype and PML

| HLA-DRB1*15:01 ancestral haplotype | | |
|---|---|---|
| | No | Yes |

| | | | | |
|---|---|---|---|---|
| Patients not destined to develop PML | 267 | (51.84) | 248 | (48.16) |
| Patients who developed PML * | 16 | (76.19) | 5 | (23.81) |

* p = 0.03

TABLE 3
Correlations (Spearman test) between the relative abundance
of Genus Phyllobacterium at T0 in blood and immunological
markers at baseline and 1-year follow-up.

| | Genus Phyllobacterium at T0 | | | | | |
|---|---|---|---|---|---|---|
| | NO DR2 | | | DR2 | | |
| | N | R | P | N | R | P |
| Baseline | | | | | | |
| IgG | 238 | 0.08073 | 0.2147 | 222 | 0.16378 | 0.0146 |
| IgA | 238 | 0.00917 | 0.8881 | 222 | 0.12029 | 0.0737 |
| IgM | 238 | 0.07702 | 0.2365 | 220 | 0.11857 | 0.0793 |
| CD4 T | 246 | −0.05709 | 0.3726 | 227 | −0.00820 | 0.9022 |
| lymphocyte | | | | | | |
| CD8 T | 245 | 0.00454 | 0.9436 | 226 | 0.15020 | 0.0239 |
| lymphocyte | | | | | | |
| CD19 T | 212 | −0.07612 | 0.2699 | 195 | 0.02313 | 0.7483 |
| lymphocyte | | | | | | |
| At 1-year | | | | | | |
| follow-up | | | | | | |
| IgG | 84 | 0.06112 | 0.5808 | 94 | 0.03658 | 0.7263 |
| IgA | 84 | 0.10463 | 0.3436 | 94 | −0.04609 | 0.6591 |
| IgM | 84 | −0.02500 | 0.8214 | 94 | 0.19737 | 0.0566 |
| CD4 T | 90 | −0.03016 | 0.7778 | 102 | 0.11863 | 0.2350 |
| lymphocyte | | | | | | |
| CD8 T | 90 | 0.09604 | 0.3679 | 102 | 0.40459 | <.0001 |
| lymphocyte | | | | | | |
| CD19 T | 87 | −0.06691 | 0.5380 | 101 | 0.14126 | 0.1588 |
| lymphocyte | | | | | | |

### REFERENCES

Throughout this application, various references describe the state of the art to which this invention pertains. The disclosures of these references are hereby incorporated by reference into the present disclosure.
1. Glaser A, Stahmann A, Meissner T, et al. Multiple sclerosis registries in Europe—An updated mapping survey. Mult Scler Relat Disord. 2018; 27: 171-178.
2. Howard J, Trevick S, Younger D S. Epidemiology of Multiple Sclerosis. Neurol Clin. 2016; 34(4): 919-939.
3. Polman C H, O'Connor P W, Havrdova E, et al. A randomized, placebo-controlled trial of natalizumab for relapsing multiple sclerosis. N Engl J Med. 2006; 354:899-910.
4. Bloomgren G, Richman S, Hotermans C, et al. Risk of natalizumab-associated progressive multifocal leukoencephalopathy. N Engl J Med. 2012; 366:1870-80.
5. Tan C S, Koralnik I J. Progressive multifocal leukoencephalopathy and other disorders caused by JC virus: clinical features and pathogenesis. Lancet Neurol 2010; 9:425-437
6. Weinshenker B G, Santrach P, Bissonet A S, et al. Major histocompatibility complex class II alleles and the course and outcome of MS: a population-based study. Neurology.

1998; 51:742-7
7. Seboun El, Robinson M A, Doolittle T H, Ciulla T A, Kindt T J, Hauser S L. A susceptibility locus for multiple sclerosis is linked to the T cell receptor beta chain complex. Cell. 1989; 57:1095-100.
8. Kiryluk K, Li Y, Sanna-Cherchi S, Rohanizadegan M, et al. Geographic differences in genetic susceptibility to IgA nephropathy: GWAS replication study and geospatial risk analysis. PLOS Genet. 2012; 8:e1002765.
9. Gourraud P A, Harbo H F, Hauser S L, Baranzini S E. The genetics of multiple sclerosis: an up-to-date review. Immunol Rev. 2012; 248:87-103.
10. Seledtsov V I, Seledtsova G V. A balance between tissue-destructive and tissue-protective immunities: a role of toll-like receptors in regulation of adaptive immunity. Immunobiology. 2012; 217:430-5
11. Madhi S A, Klugman K P; Vaccine Trialist Group. A role for Streptococcus pneumoniae in virus-associated pneumonia. Nat Med. 2004; 10:811-3
12. Hartwig S M, Ketterer M, Apicella M A, Varga S M. Non-typeable Haemophilus influenzae protects human airway epithelial cells from a subsequent respiratory syncytial virus challenge. Virology. 2016; 498:128-135.
13. Outteryck O, Ongagna J C, Brochet B, et al. A prospective observational post-marketing study of natalizumab-treated multiple sclerosis patients: clinical, radiological and biological features and adverse events. The BIONAT cohort. Eur J Neurol 2014; 21:40-48.
14. Dong-Si T, Richman S, Wattjes M P, et al. Outcome and survival of asymptomatic PML in natalizumab-treated MS patients. Ann Clin Translational Neurol. 2014; 1:755-764
15. Lluch J, Servant F, Paisse S, et al. The Characterization of Novel Tissue Microbiota Using an Optimized 16S Metagenomic Sequencing Pipeline. PLOS One. 2015; 10:e0142334.
16. Paisse S, Valle C, Servant F, et al. Comprehensive description of blood microbiome from healthy donors assessed by 16S targeted metagenomic sequencing. Transfusion. 2016; 56:1138-1147.
17. Anhê, F. F., Jensen, B. A. H., Varin, T. V. et al. Type 2 diabetes influences bacterial tissue compartmentalisation in human obesity. Nat Metab 2000; 2, 233-242.
18. Mangalam A K, Taneja V, David C S. HLA class II molecules influence susceptibility versus protection in inflammatory diseases by determining the cytokine profile. J Immunol. 2013; 190:513-8.
19. Sarri C A, Markantoni M, Stamatis C, et al; MALWEST project. Genetic Contribution of MHC Class II Genes in Susceptibility to West Nile Virus Infection. PLOS One. 2016; 11(11):e0165952
20. Dunstan S J, Hue N T, Han B, et al. Variation at HLA-DRB1 is associated with resistance to enteric fever. Nat Genet. 2014; 46:1333-6
21. Mantelin, S; Saux, M. F.; Zakhia, F, et al. Emended description of the genus Phyllobacterium and description of four novel species associated with plant roots: Phyllobacterium bourgognense sp. nov., Phyllobacterium ifriqiyense sp. nov., Phyllobacterium leguminum sp. nov. And Phyllobacterium brassicacearum sp. nov″. International Journal of Systematic and Evolutionary Microbiology. 2006; 56: 827-39.
22. Guo C, Li Y, Wang P, et al. Alterations of Gut Microbiota in cholestatic infants and their Correlation with hepatic function. Front Microbiol. 2018; 9:2682.
23. Huang Y J, Kim E, Cox M J et al. A persistent and diverse airway microbiota present during chronic obstructive pulmonary disease exacerbations. OMICS. 2010; 14:9-59.
24. Boutin S, Depner M, Stahl M. et al Comparison of Oropharyngeal Microbiota from Children with Asthma and Cystic Fibrosis. Mediators Inflamm. 2017; 2017:5047403.
25. Boutin S, Graeber S Y, Weitnauer M, et al. Comparison of microbiomes from different niches of upper and lower airways in children and adolescents with cystic fibrosis. PLOS One. 2015; 10:e0116029
26. Ferreira R M, Pereira-Marques J, Pinto-Ribeiro I, Costa J L, Carneiro F, Machado J C, Figueiredo C. Gastric microbial community profiling reveals a dysbiotic cancer-associated microbiota. Gut. 2018; 67:226-236.
27. Barr-Beare E, Saxena V, Hilt E E, Thomas-White K, Schober M, Li B, Becknell B, Hains D S, Wolfe A J, Schwaderer A L. The Interaction between Enterobacteriaceae and Calcium Oxalate Deposits. PLOS One. 2015; 10:e0139575.
28. Torrazza R M, Ukhanova M, Wang X, Sharma R, Hudak M L, Neu J, Mai V. Intestinal microbial ecology and environmental factors affecting necrotizing enterocolitis. PLOS One. 2013; 8:e83304.
29. Price D A, Birtles R J, Levine T S, Main J, Coker R J. Bacillary angiomatosis: a role for Phyllobacterium? AIDS. 1997; 11:1186-7
30. Erickson A K, Jesudhasan P R, Mayer M J, Narbad A, Winter S E, Pfeiffer J K. Bacteria Facilitate Enteric Virus Co-infection of Mammalian Cells and Promote Genetic Recombination. Cell Host Microbe. 2018; 23:77-88.e5.
31. Jones M K, Watanabe M, Zhu S, et al. Enteric bacteria promote human and mouse norovirus infection of B cells. Science. 2014; 346:755-9.
32. Laghi L, Randolph A E, Chauhan D P, et al. JC virus DNA is present in the mucosa of the human colon and in colorectal cancers. Proc Natl Acad Sci USA. 1999; 96:7484-9.
33. Ricciardiello L, Laghi L, Ramamirtham P, et al. JC virus DNA sequences are frequently present in the human upper and lower gastrointestinal tract. Gastroenterology. 2000; 119:1228-35
34. Almand E A, Moore M D, Jaykus L A. Virus-Bacteria Interactions: An Emerging Topic in Human Infection. Viruses. 2017; 9:58.
35. Kuss S. K., Best G. T., Etheredge C. A. et al. Intestinal microbiota promote enteric virus replication and systemic pathogenesis. Science. 2011; 334:249-252.
36. Robinson C. M., Jesudhasan P. R., Pfeiffer J. K. Bacterial Lipopolysaccharide Binding Enhances Virion Stability and Promotes Environmental Fitness of an Enteric Virus. Cell Host Microbe. 2014; 15:36-46.
37. Selgrad M, De Giorgio R, Fini L. et al. JC virus infects the enteric glia of patients with chronic idiopathic intestinal pseudo-obstruction. Gut. 2009; 58:25-32.
38. Plavina T, Subramanyam M, Bloomgren G, et al. Anti-JC virus antibody levels in serum or plasma further define risk of natalizumab-associated progressive multifocal leukoencephalopathy. Ann Neurol. 2014; 76:802-812.
39. Vukusic S, Rollot F, Case R, et al. Progressive Multifocal Leukoencephalopathy Incidence and Risk Stratification Among Natalizumab Users in France. JAMA Neurol 2019; 77: 94-102.
40. Kappos L, Bates D, Edan G, et al. Natalizumab treatment for multiple sclerosis: updated recommendations for patient selection and monitoring. Lancet Neurol 2011; 10:745-758.

1. A method of treating a patient with a VLA-4 antagonist comprising,
i) determining the abundance of Phyllobacterium in a biological sample obtained from the patient, and
ii) administering a therapeutically effective amount of the VLA-4 antagonist to a patient identified as having an abundance of Phyllobacterium that is lower than a corresponding reference value.

2. The method of claim 1 wherein the patient has not previously been treated with the VLA-4 antagonist.

3. The method of claim 1 wherein the patient suffers from multiple sclerosis.

4. The method of claim 1 further comprising determining if the patient harbours a HLA-DR2 haplotype.

5. The method of claim 1 wherein the patient harbors a HLA-DR2 haplotype.

6. The method of claim 5 wherein the HLA-DR2 haplotype is selected from the group consisting of DRB1*1501, DRB1*15021, DQA102 and a DW2 haplotypes.

7. The method of claim 1 wherein the VLA-4 antagonist is an antibody that blocks VLA-4 activation.

8. The method of claim 1 wherein the VLA-4 antagonist is Natalizumab.

9. The method of claim 1 wherein the biological sample is a blood sample.

10. The method of claim 1 further comprising comparing the determined abundance with a predetermined reference value wherein a differential between said determined abundance and said predetermined reference value indicates whether or not the patient has or is at risk of having a PML.

11. (canceled)

12. The method of claim 1 further comprising comparing the determined abundance with a predetermined reference value and concluding that the patient has or is at risk of having a PML when the abundance determined at step i) is higher than the predetermined reference value and when the patient does not harbour a DRB1*1501 haplotype.

13. The method of claim 1 further comprising calculating a score and comparing the score to a predetermined reference value wherein a difference between said score and said predetermined reference value indicates whether the patient has or is at risk of having a PML.

14. (canceled)

15. (canceled)

16. The method of claim 1, wherein the patient has or is at risk of having a progressive multifocal leukoencephalopathy.

17. The method of claim 1 wherein the patient is already being treated with the VLA-4 antagonist.
